(19) United States
(12) Patent Application Publication
(10) Pub. No.: US 2008/0228105 A1
(43) Pub. Date: Sep. 18, 2008

(54) Sampling Means

(75) Inventors: Todd Michael Howell, Auckland (NZ); Gerard Date Willem Boterman, Auckland (NZ)

Correspondence Address: HENRICKS SLAVIN AND HOLMES LLP;SUITE 200, 840 APOLLO STREET, EL SEGUNDO, CA, 90245, US

(73) Assignee: TAGAM LIMITED

(21) Appl. No.: 11/996,799

(22) PCT Filed: Jul. 25, 2006
(86) PCT No.: PCT/NZ2006/000190

§ 371 Date: Apr. 24, 2008

(30) Foreign Application Priority Data
Jul. 25, 2005 (NZ) 541467

(51) Int. Cl. A61B 10/02

(52) U.S. Cl. 600/567

(57) ABSTRACT

Sampling Means A sampling means for obtaining an organic sample (85) from an animal. The sampling means includes a punch means (18) adapted to be securable in use with a first part (70) of an applicator (100). A housing means (8) is adapted in use to be accommodated, or provided, by a second part (2) of the applicator (100). Operation of the applicator (100) in use being adapted to move said punch means (18) through a required part (80) of an animal to extract a sample (85) thereof. The punch means (18) being receivable within said housing means (8) to enable said housing means (8), with said punch means (18) and said sample (85), to be removable from said second part (2) of said applicator (100), and wherein the sampling means is not associated with an animal tag.

The present invention relates to improvements in or relating to sampling means and more particularly but not exclusively to sampling means for obtaining an organic sample from an animal for the purposes of its analysis and its use in uniquely identifying the animal.

In New Zealand and elsewhere various databases have been created requiring a unique identifier of each animal registered. Although various distinguishable characteristics can be used, the provision of an organic sample, in particular tissue, for DNA testing is one of the preferred options.

To the present time various proposals have been put forward whereby an organic sample is taken from an animal which is then retained with the ear tag which is attached to the animal or an animal is tagged simultaneously with a sampling operation and the sample container is removable from the tag, as disclosed in U.S. Pat. No. 6,509,187.

However, the linking of the tagging and sampling operations has been found to have disadvantages including when tagging of an animal is not required.

### OBJECTS OF THE INVENTION

It is an object of the present invention to overcome or at least alleviate problems with sampling means or methods available at the present time and/or at least to provide the public with a useful choice.

Further objects will become apparent from the following description.

### SUMMARY OF THE INVENTION

According to a first aspect of the present invention there is provided a sampling means for obtaining an organic sample from an animal and including a punch means adapted to be securable in use with a first part of an applicator, a housing means adapted in use to be accommodated, or provided, by a second part of the applicator, operation of said applicator in use being adapted to move said punch means through a required part of an animal to extract a sample therefore, said punch means being receivable within said housing means to enable said housing means, with said punch means and said sample, to be removable from said second part of said applicator, and wherein the sampling means is not associated with an animal tag.

According to a second aspect of the present invention, there is provided a sampling means for obtaining an organic sample from an animal, the sampling means comprising separate punch means and housing means, the punch means being adapted to be moved completely through a part of an animal to obtain a sample therefrom and the punch means and the housing means being engageable with each other to contain the sample in the housing means, and wherein the sampling means is not associated with an animal tag.

Typically, the punch means is adapted to be mounted on a first part of an applicator and the housing means is adapted to be mounted on a second part of an applicator, and operation of the applicator causes the punch means to be moved through the part of the animal and to engage with the housing means.

The sampling means may further comprise a removable holding means which is located in said second part of the applicator and the housing means is located in the removable holding means.

According to a third aspect of the present invention a method of sampling to provide a sample of organic material from an animal includes:
(i) providing a punch means adapted to be moveable by a first part of an applicator through part of an animal to obtain a sample;
(ii) providing a housing means adapted to be accommodated by a second part of an applicator;
(iii) adapting said punch means and said housing means to be engageable together so that the punch means with said sample may be located within said housing means and wherein said housing means and said punch means are adapted to be removable from said second part of said applicator.

According to a fourth aspect of the present invention, there is provided a sampling method for obtaining a sample of organic material from an animal, the method comprising
(i) mounting a punch means on a first part of an applicator;
(ii) mounting a housing means on a second part of an applicator;
(iii) inserting a part of an animal between the first and second parts of the applicator;
(iv) activating the applicator to cause the punch means to penetrate and pass through the part of the animal to take a sample from the part of the animal, and to engage the housing means so that the sample is located in the housing means; and
   wherein the sampling method does not occur simultaneously with the application of a tag to the part of the animal.

Optionally said method further provides the removing of a holding means from the second part of the applicator, in which said housing mean is locatable.

Preferably, the punch means comprises an elongate body. Typically, the elongate body has a recess which engages with the first part of the applicator. Preferably, the elongate body and the recess are configured such that the elongate body prevents or minimises contact between the part of the animal and the first part of the applicator and typically, prevents or minimises contact between the first part of the applicator and any body fluids of the animal exposed by the sampling method. Typically, the first part of the applicator is a pin which locates in a recess in the punch means.

Typically, the punch means includes a sample chamber. Preferably, the sample chamber is in the form of a recess in the punch means. It is possible that more than one sample chamber may be provided on the punch means.

Preferably, one of the housing means and the punch means has an openable closure that permits access to the sample chamber through the closure, for example, to enable testing of a sample collected in the chamber. Typically, the closure comprises a deformable member. Preferably the deformable member is elastically deformable so that the closure is self-closing after access to the sample.

Preferably, the openable closure comprises two deformable members.

Typically, the two deformable members engage each other when the closure is closed and at least partially separate when the closure is open.

Preferably, the deformable member is inclined towards the sample chamber.

Typically, the openable closure is in the housing means and preferably, at an opposite end of the housing means from where the punch means engages with the housing means.

Preferably, the closure is openable by a DNA testing probe.

Preferably, the other of the housing means and the punch means comprises a portion adapted to engage with a laboratory tray, such as a DNA testing tray.

According to a further aspect of the present invention a sampling means and/or sampling method is substantially as herein described/or as illustrated in the accompanying drawings.

Further aspects of this invention which should be considered in all its novel aspects will become apparent from the following description given by way of example of possible embodiments thereof.

### BRIEF DESCRIPTION OF DRAWINGS

FIG. 1 is a perspective view of an applicator with a punch, an adaptor and a housing mounted on the applicator prior to a sampling operation.

FIG. 2 is a plan view of the punch viewed from an opposite end from a tip of the punch.

FIG. 3 is a perspective view from the side and above of the punch;

FIG. 4 is a side view of the punch;

FIG. 5 is a side of the punch similar to FIG. 4 but with the punch rotated through 90°;

FIG. 6 is a top view of the housing;

FIG. 7 is a perspective view of the housing;

FIG. 8 is a first side view of the housing;

FIG. 9 is a second side view of the housing with the housing rotated 90° compared to FIG. 8;

FIG. 10 is a cross-sectional view through the housing;

FIG. 11 is an enlarged cross-sectional view of the part indicated as “A” in FIG. 10;

FIG. 12 is a perspective view from the side and above of the adaptor;

FIG. 13 is a plan view of the adaptor;

FIG. 14 is a perspective view from the side and below of the adaptor;

FIG. 15 is a side view of the adaptor;

FIG. 16 is a perspective view from the side and above after a sample has been taken with the punch engaged with the housing and the housing in the adaptor;

FIG. 17 is a plan view of the punch, the housing and the adaptor shown in FIG. 16;

FIG. 18 is a perspective view from the side and below of the punch, the housing and the adaptor shown in FIG. 16;

FIG. 19 is a side view of the punch, the housing and adaptor shown in FIG. 16;

FIG. 20 is a partial cross-sectional view of the applicator in a first position with the punch, the adaptor and the housing mounted in the applicator prior to a sample being taken;

FIG. 21 is a partial cross-sectional view of the applicator similar to that shown in FIG. 20 but with an ear of an animal inserted into the applicator;

FIG. 22 is a partial cross-sectional view of the applicator showing the applicator in a second position during a sampling application;

FIG. 23 is a partial cross-sectional view of the applicator showing the applicator in a third position during a sampling operation;

FIG. 24 is a partial cross-sectional view through the applicator with the applicator shown in a fourth position during a sampling operation;

FIG. 25 is a partial cross-sectional view through the applicator showing the applicator returning to the first position after a sample has been taken; and

FIG. 26 is a cross-sectional view showing the punch and the housing located in a laboratory tray after a sample has been taken and during a DNA testing operation.

### BRIEF DESCRIPTION OF POSSIBLE EMBODIMENTS

The present invention enables the speedy and effective taking of an organic sample from an animal for its subsequent analysis. The analysis could be DNA testing to provide a unique identification of the animal, such as in a registration database. However, alternatively or in addition, the analysis could be testing for disease or any other desired purpose.

To facilitate the sample being taken, the present invention may use a well known type of ear tag applicator subject to any appropriate modification.

One such applicator is the well known Zee Tags™ applicator which is described in New Zealand patent specification no: 335702 which specification is incorporated herein in its entirety where appropriate by way of reference. That applicator is usually used in the application of two piece tags to an animal's ear. The applicator has two arms which are pivotally connected together so that with a male tag positioned on an applicator pin, the male tag is adapted to be inserted through an animal's ear to engage with a female tag held on the opposing second arm of the applicator. The first and second arms of the applicator are then adapted to spring apart at the appropriate time leaving the tag in the animal's ear and providing the appropriate identification.

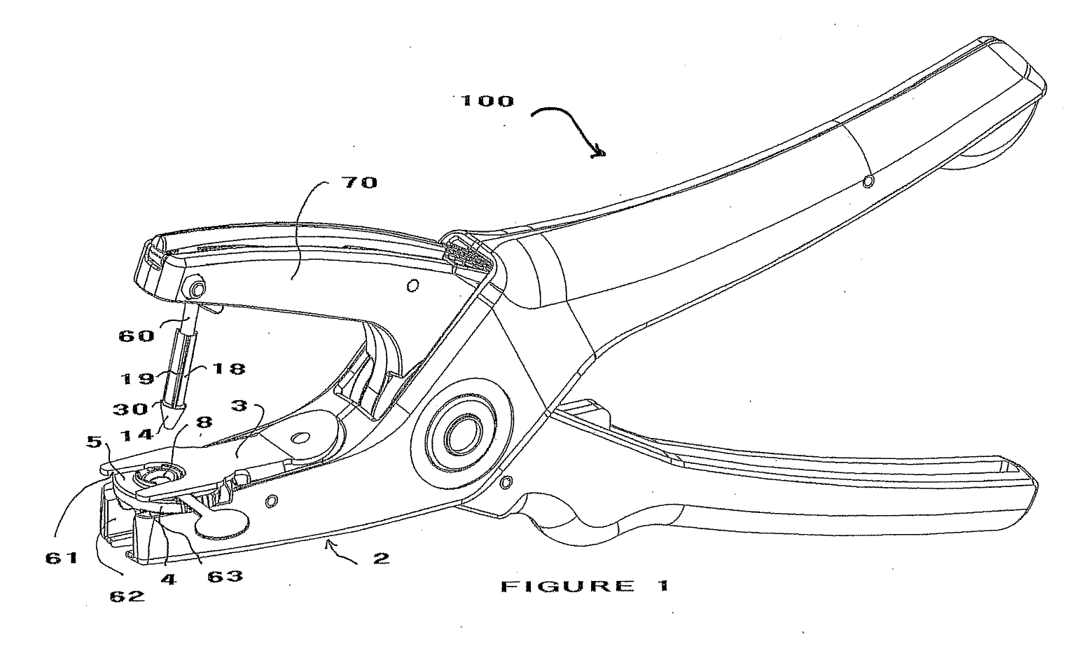

FIG. 1 shows an applicator 100 which includes a first arm 2 having a clamp plate 3 and a second arm 70 with a pin 60. The pin 60 extends from the second arm 70 towards the first arm 2 and towards recess 61 in the clamping plate 3 and recess 62 in the second arm 2. The applicator 100 is conventionally used for applying identification tags to animals. When applying identification tags to animals, a female portion of the tag is located between the clamping plate 3 and the first arm 2 and retained in position by the clamping plate 3. A male portion of the tag is mounted on the pin 60.

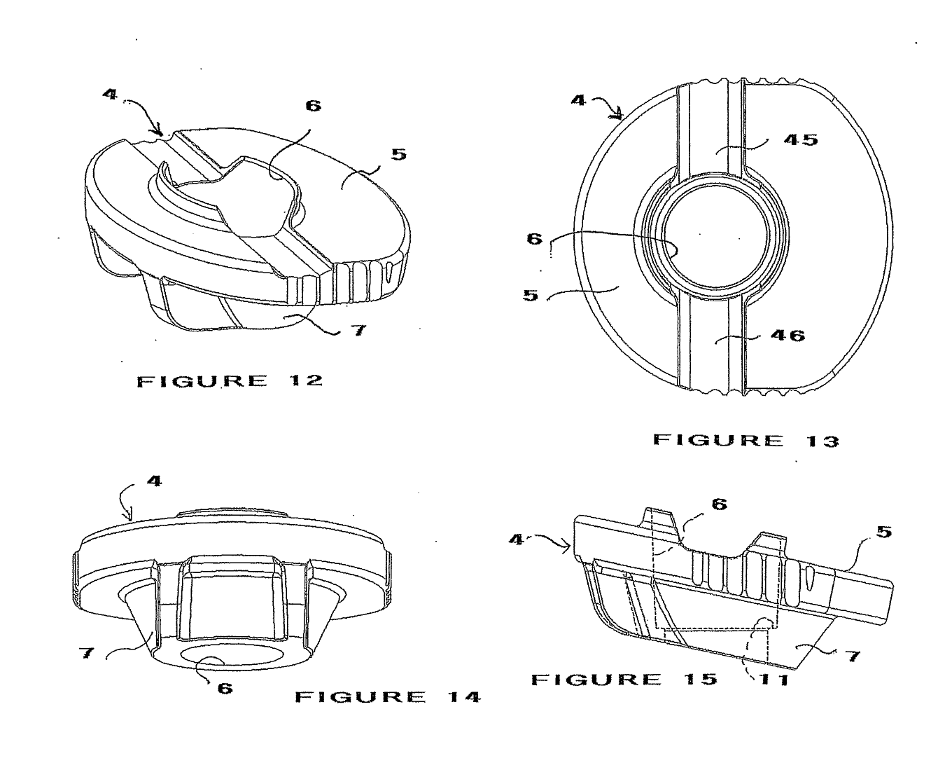

However, in order to take an organic sample from an animal, the female portion of the tag is replaced by an adaptor 4, a housing 8 is located within an opening 6 (see FIGS. 12 to 15) in the adaptor 4 and a punch 18 is mounted on the pin 60.

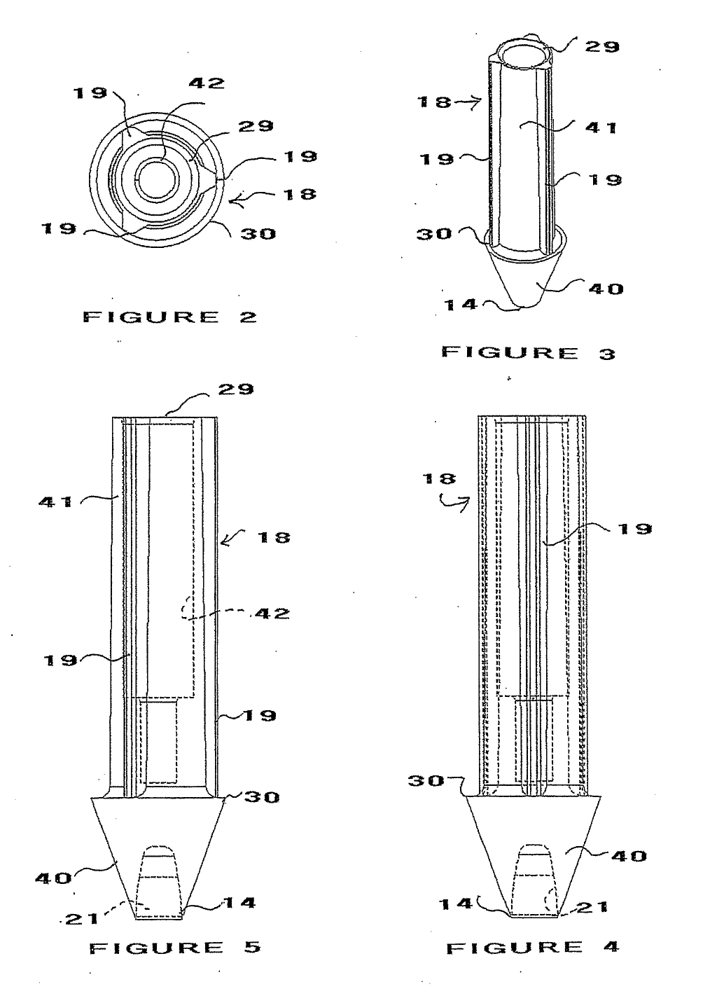

FIGS. 2 to 5 show the punch 18 in more detail. As shown in FIGS. 2 to 5, the punch 18 has a tip portion 40 adapted to be able to pass through a required part of an animal for collecting a suitable sample of tissue or other organic matter. Tip 40 is of a generally frusto-conical shape and has a recess 21 at end 14 the tip 40. The opposite end of the tip 40 defines an edge 30. Extending from end 29 of the punch 18 to the edge 30 of the tip 40 are three ribbed sections 19 that are spaced equidistantly around main body section 41 of the punch 18. The body section 41 is hollow and defines a recess 42 that is adapted to receive the pin 60. The punch 18 maybe manufactured from any suitable material and could, for example, be manufactured from a plastics material using a moulding process. Although the ribbed sections 19 are typically spaced equidistantly around the body section 41, it is possible that they may not be equidistantly spaced.

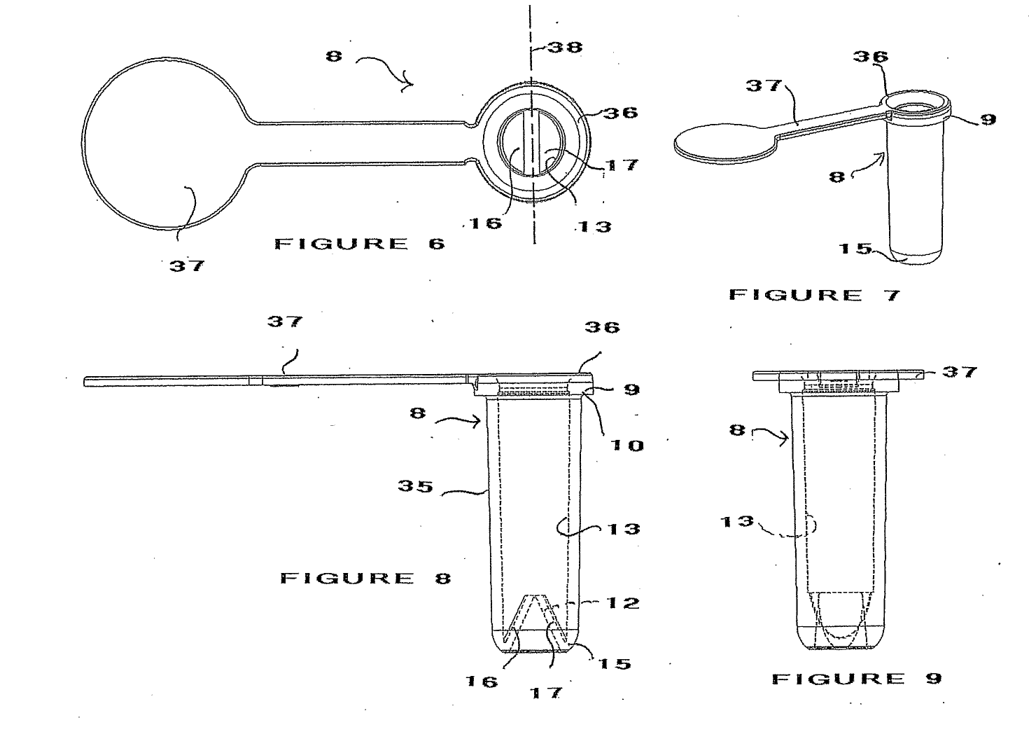

FIGS. 6 to 9 show the housing 8 in more detail. The housing 8 has a body section 35 which is generally tubular and defines an internal chamber 13. At end 36 of the body section 35, there is a flange 9 which has a bottom edge 10. In addition, an elongate flat portion 37 extends from the flange 9 and may be used as a handle or a grip for handling the housing 8. The flat portion 37 may be designed to be broken off from the flange to enable it to be easily removed. In addition, or alternatively the flat portion 37 may be used for carrying a code (for example a bar code) and/or indicia to enable a sample and/or an animal to be identified. The code and/or indicia can be applied to the flat portion 37 and the body section 35 by printing directly onto the surfaces or by an adhesive label. Typically, the same code and/or indicia are also applied externally to the body section 35 of the housing so that the housing 8 still carries the code and/or indicia after the flat portion 37 is removed from the flange 9. At the other end 15 of the body section 35 is a closure 12.

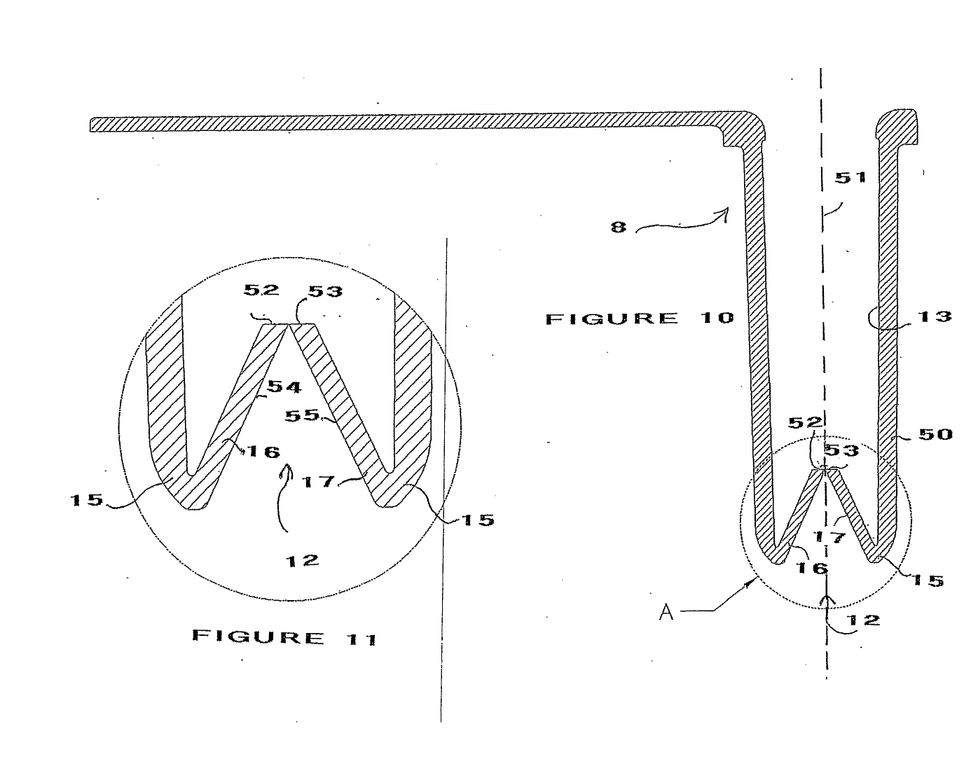

The closure 12 is openable and is shown in more detail in FIGS. 10 and 11. The closure 12 comprises two members 16, 17 that extend from the end 15 of the body section 35 inwardly towards diameter 38 of the body section 35 and inwardly along longitudinal axis 51 into the chamber 13 so that sides 54, 55 of the members 16, 17, respectively, which are outside the chamber 13, define in cross-section a generally “V” shape. It can be seen that free ends 52, 53 of the members 16, 17, respectively, butt against each other to close the closure 12. However, if a suitable object is inserted into the V shape defined by the sides 54, 55 with sufficient force, the members 16, 17 deform so that the free ends 52, 53 separate from each other to create an opening in the closure 12 into the chamber 13.

The housing 8 can be manufactured from any suitable material, and as with the punch 18, it could be manufactured, for example, from a plastics material, such as by a moulding process.

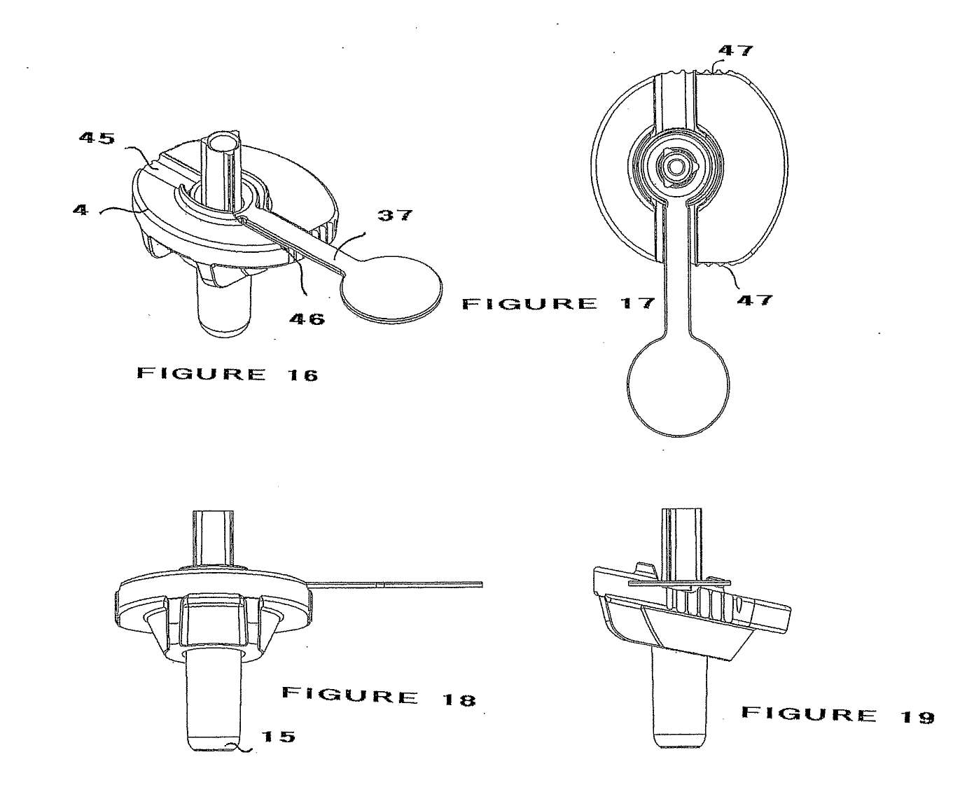

The adaptor 4 is shown in more detail in FIGS. 12 to 15. The adaptor 4 has a flange 5 that defines an opening 6 which extends through the flange 5 and through a boss portion 7 located below the flange 5. The opening 6 is configured to receive the body section 35 of the housing 8 and the opening 6 has a shoulder 11 which can be engaged by the edge 10 on the flange 9. When the housing 8 is inserted into the adaptor 4, the body section 35 extends through the opening 6 the housing 8 can be rotated relative to the adaptor 4 such that the flat portion 37 of the housing 8 is located in either of two recesses 45, 46 in the flange 5 which extend radially outwardly from the opening 6 and diametrically opposite each other. FIGS. 16 to 19 show how the flat portion 37 locates in the recesses 46 when the housing is inserted into the adaptor 4. In a similar manner, the flat portion can, alternatively, be located in the recess 45.

In use, pin 60 is inserted into the opening in the end 29 of the punch 18 so that the pin 60 is located in the recess 42. The clamp plate 3 is lifted and the adaptor 4 is inserted into the arm 2 so that the flange 5 is located between the plate 3 and the top surface 63 of the arm 2 and so that the boss 7 locates in the recess 62 in the arm 2. The housing 8 may be inserted into the adapter 4 prior to insertion of the adaptor 4 into the arm 2. However, alternatively, using grooved sections 47 on the edge of the flange 5, it is possible for an operator to grip the adaptor 4 and rotate it through 90° in the plane of the flange 5 so that one of the recesses 45, 46 is aligned in the recess 61 of the clamping plate 3. This enables the housing 8 to be inserted into the adaptor 4 with the flat portion 37 located in the recess 45, 46 that is located in the recess 61. When the housing 8 is located in the adaptor 4, the adaptor 4 can then rotated using grooved sections 47 to the position shown in FIG. 1.

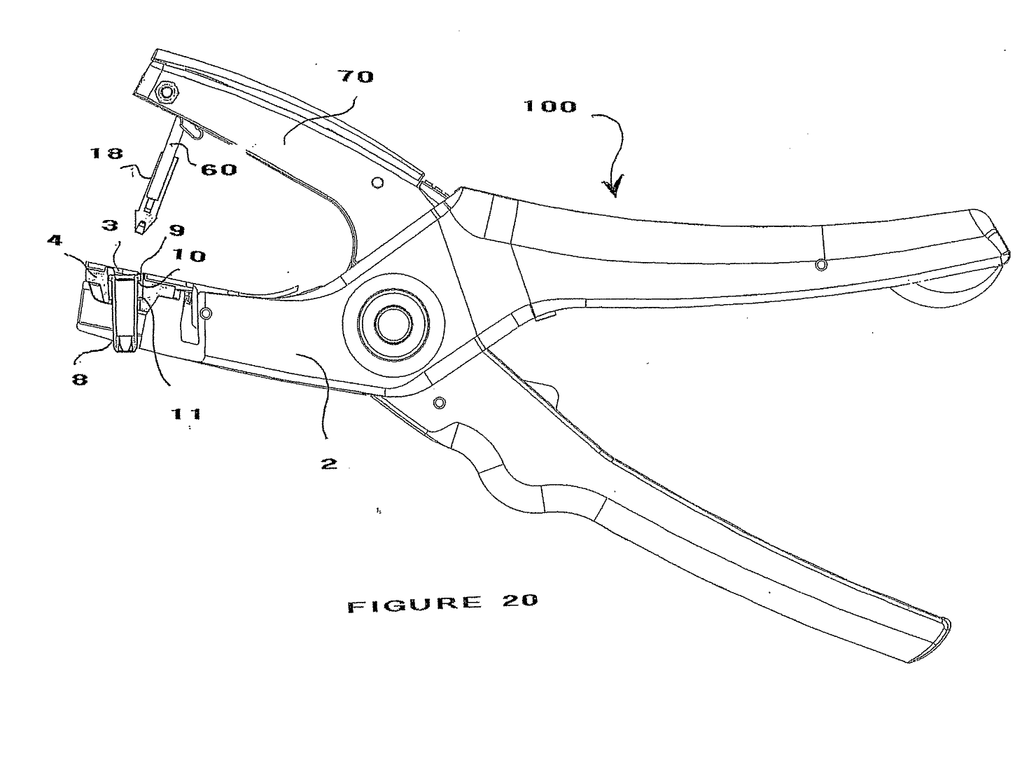

FIG. 20 is a side view of the applicator 100 in the position of FIG. 1 but with the punch 18, the adaptor 4, the housing 8 and part of the first arm 2 shown in cross-section. From this it can be seen that prior to taking the sample from part of an animal the housing 8 is located in the adaptor 4 such that the bottom edge 10 of the flange 9 is spaced from the shoulder 11 of the adaptor 4.

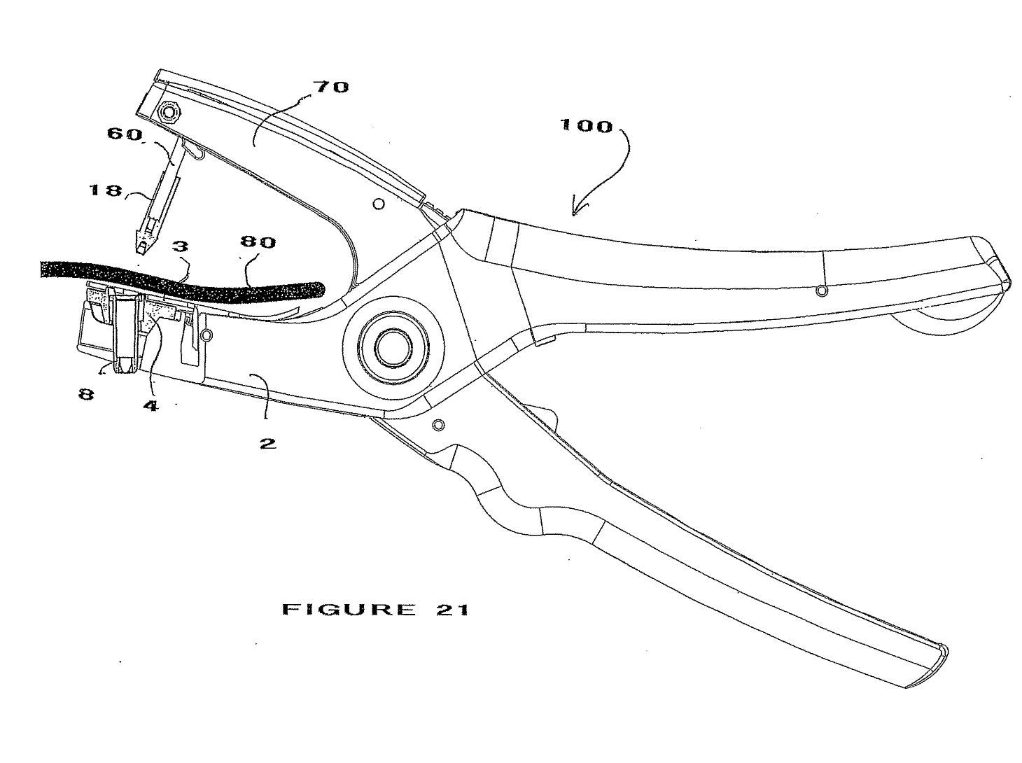

After the punch 18, the housing 8 and the adaptor 4 are mounted on the applicator 100, part of an animal, which is typically an ear 80, is inserted into the applicator between the first and second arms 2, 70, as shown in FIG. 21.

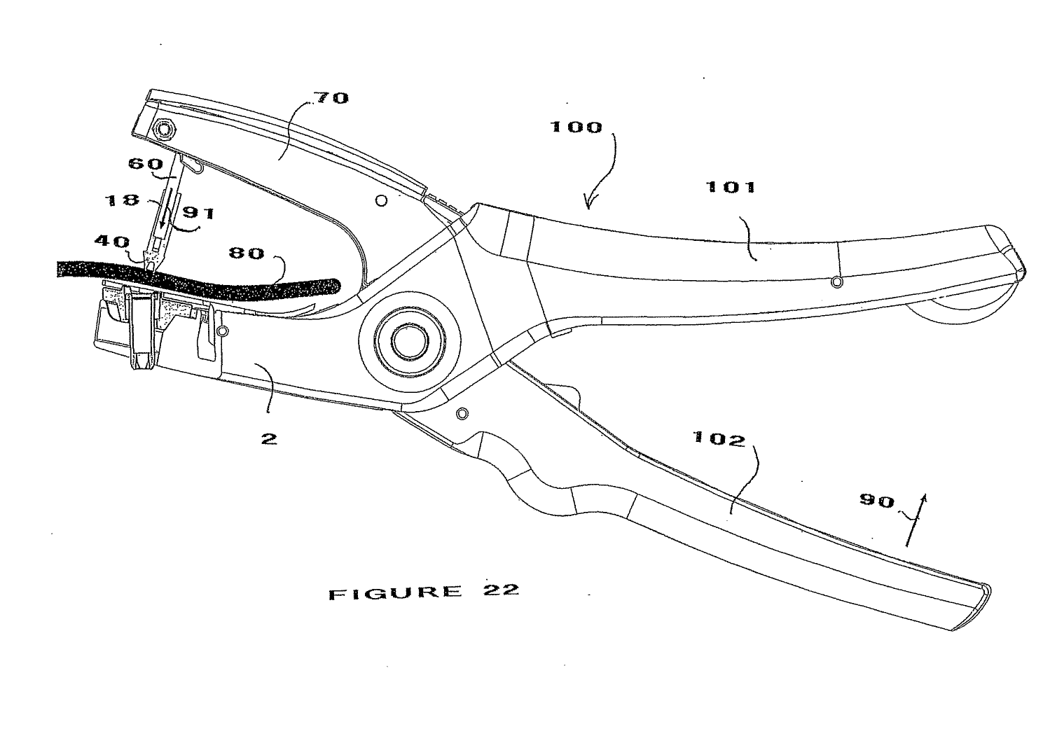

After insertion of the ear 80, grip sections 101, 102 are gripped by an operator and pressed together, as indicated by arrow 90 in FIG. 22. This causes the first and second arms 2, 70 to move towards each other and the pin 60 with the punch 18 mounted on it to move in the direction of arrow 91 towards the ear 80 until end 14 of the tip 40 engages with the ear 80.

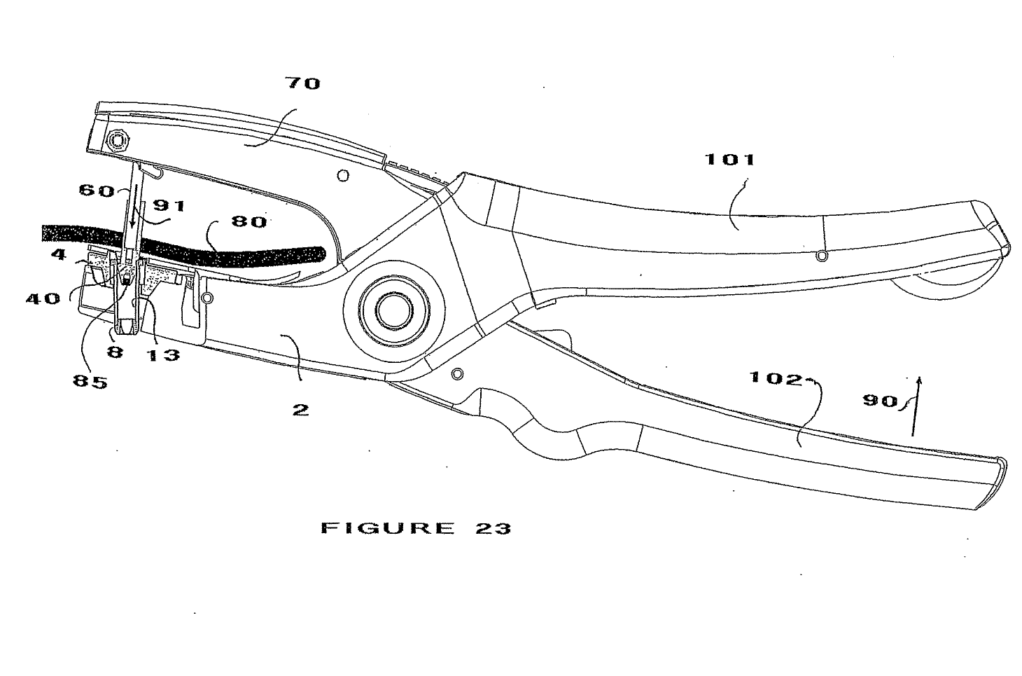

Further movement of the grips 101, 102 in the direction of arrow 90 causes the tip 40 to pass through the ear 80 and for the tip 40 to enter into the chamber 13 of the housing 8 through the opening in the end 36, as shown in FIG. 23. It can also be shown seen from FIG. 23 that as the tip 40 passed through the ear 80 a section of tissue 85 was removed from the ear 80 and retained in the recess 21 in the tip 40. If it is desired to collect more than one sample from an animal, it is possible that the tip 40 could include two or more recesses.

The tip 40 of the punch 18 and the body section 35 of the housing 8 are configured so that the tip 40 is an interference fit or friction fit in the chamber 13. This may be achieved, for example, by designing the body section 35 and the tip 40 so that the internal diameter of the chamber 13 is less than the diameter of the edge 30 and the tip 40 and/or the body section 35 are designed to be deformable so that the edge 30 can enter the chamber 13 but so that the tip 40 resists removal from the chamber 13.

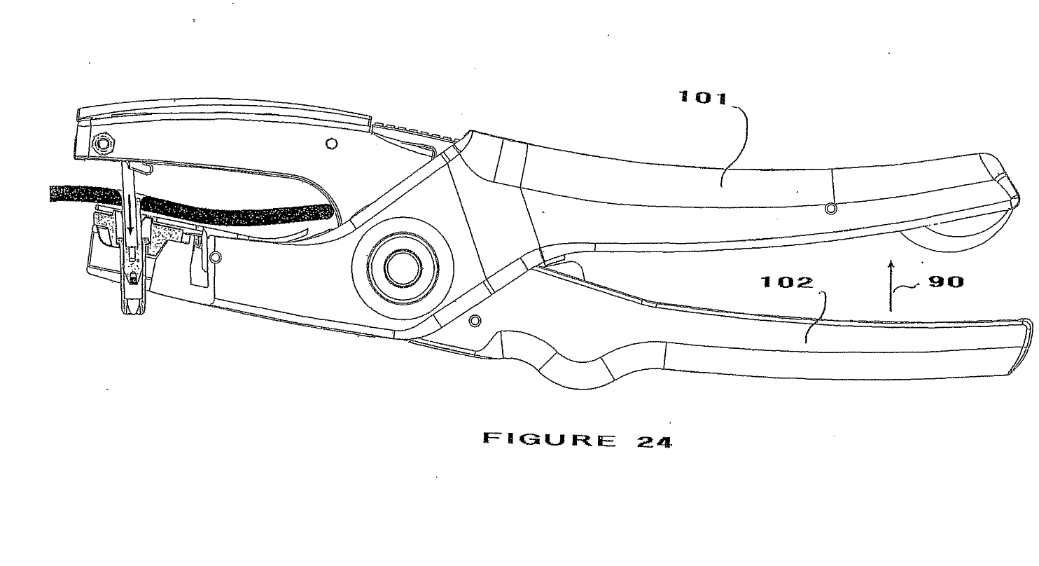

Further movement of the grips 101, 102 in the direction of the arrow 90 drives the first and second arms 2, 70 further together and drives the housing 8 further into the adaptor 4 until the bottom edge 10 of the flange 9 butts against the shoulder 11 in the opening 6 and the punch 18, is pushed further into the chamber 13 so that the punch 18 is securely located in the chamber 13 by edge 30 of the tip 40 engaging with the internal side-walls of the chamber 13. The edge 30 also acts as a seal to minimise the risk of the sample becoming contaminated. This position is shown in FIG. 24. When the punch 18 is pushed into the housing 8 to the position shown in FIG. 24, the rib portions 19 help to centralise the punch 18 within the housing 8 and to reduce likelihood of any pivoting of the punch 18 relative to the housing 8, which could potentially break the seal formed by the edge 30 and/or result in the punch 18 becoming detached from the housing 8. In addition, when the punch 18 is in the position shown in FIG. 24, the body section 41 is sufficiently long that it still acts as a barrier between the ear 80 and the pin 60 to prevent or minimise the possibility of contamination of the pin 60 by the ear 80. This has the advantage of helping to prevent to the possibility of cross-infection or cross-contamination between animals.

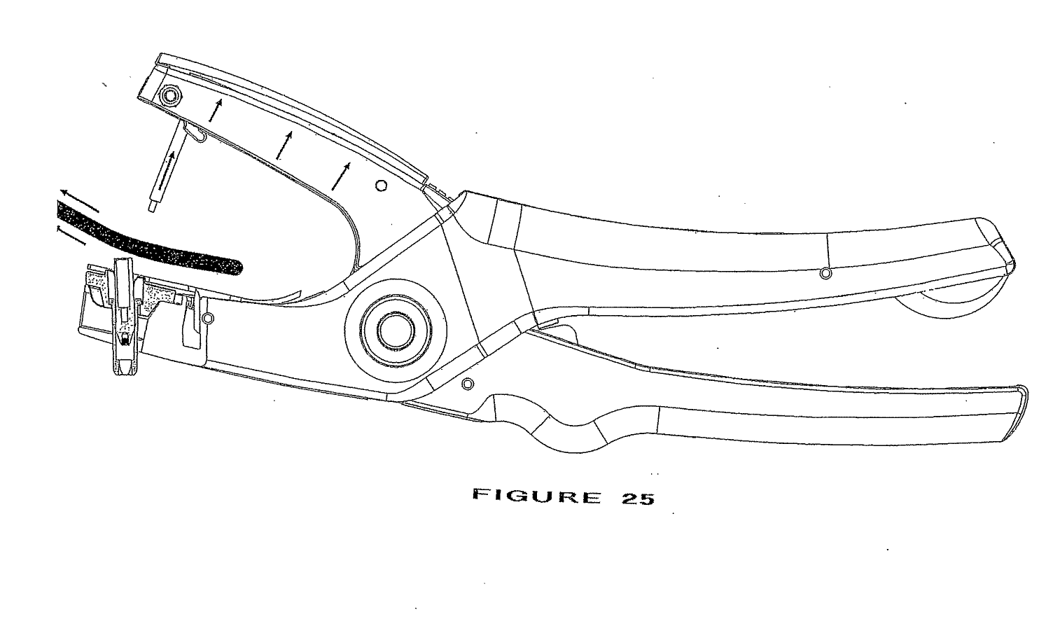

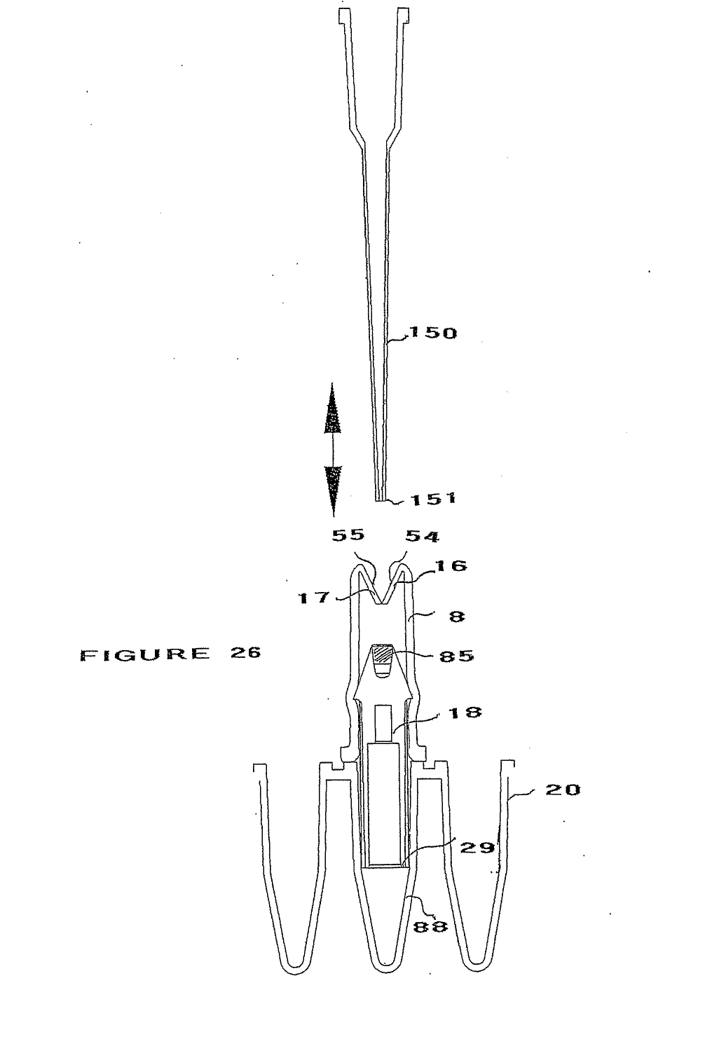

When the position shown in FIG. 24 is reached, the applicator 100 is designed to spring apart to avoid tearing the animal's ear 80, as shown in FIG. 25. This enables the immediate withdrawal of the ear 80 from between the first and second arms 2, 70 after the sample 85 has been taken.

After the sample 85 is taken from the ear 80, an operator can remove the housing 8 and punch 18 from the adaptor 4 either by rotating the adaptor through 90° then removing the housing 8 and punch 18 using the flat portion 37 as a handle as to facilitate removal or by lifting the clamp plate 3 to completely remove the adaptor 4 together with the housing 8 and punch 18.

After the housing 8 and punch 18 are removed from the adaptor 4, DNA testing can be carried out on the sample 85. In order to perform the DNA testing, the end 29 of the punch 18 is inserted into a sample recess 88 in a laboratory tray 20 so that the end 36 of the housing 8 sits at the top of the sample recess 88 and the body section 41 of the punch 18 extends into the recess 88. However, before the combined punch and housing is inserted into the laboratory tray 20 the flat portion 37 is typically removed from the flange 9. Although the flat portion 37 is removed the combined punch 18 and housing 8, the code and/or indicia are also on the body section 35 for identification of the sample or animal from which the sample was taken. When the combined punch and housing are in position in the laboratory tray 20, a pipette 150 can be inserted into the V shape formed by the surfaces 54, 55 of the members 16, 17 such that end 151 deforms the members 16,17 so that free ends 52,53 separate and permit the end 151 to pass between the free ends 52, 53 to enter the chamber 13.

Typically, in a DNA testing process the pipette 150 is used to introduce a liquid into the chamber 13 using the pipette. Removal of the pipette 150 from the chamber 13 and from between the members 16, 17 permits the members 16, 17 to revert back to the position shown in FIG. 26. The DNA testing process then typically involves heating the sample and liquid which causes the tissue of the sample 85 to be broken down and mix with the liquid. Another pipette (not shown) is then inserted through the closure 12, in the same manner as the pipette 150, to extract some of the liquid, which now contains DNA from the sample 85, for testing.

The use of a tray 20 or the like with a plurality of compartments will of course facilitate the sampling of an appropriate number of animals at one time before the individual samples are tested and the identifying data linked with a specific animal from which that particular sample has been taken.

It is thus seen that the present invention facilitates the obtaining of an organic sample 85 from an animal and the retention of the sampling means 8, 18 in the applicator 100 for its subsequent removal and testing.

It will be appreciated that there is a significant difference between the present invention and earlier proposals which combined a tagging and sampling operation. In particular, the punch means 18 of the present invention passes completely through the portion of the animal, usually the animal's ear, from which the sample is being taken. Moreover the punch means 18 is adapted to be a direct fit onto a suitable applicator pin 60 and as one punch means 18 is used per sampling, there will be no, or minimal, risk of any cross contamination between animals as the applicator pin 60 will be protected by the punch means 18.

Additionally, the provision of a removable adaptor 4 facilitates the use of an otherwise standard applicator although it is envisaged that an applicator 100 could be specifically provided for the purposes of the present invention in which case the adaptor may be a permanent holder as opposed to a removable adaptor 4.

It is appreciated therefore that unlike in other methods, nothing is left in the animal's ear, or other body part, such as a male tag.

Where in the foregoing description, reference has been made to specific components or integers of the invention having known equivalents then such equivalents are herein incorporated as if individually set forth.

Although this invention has been described by way of example and with reference to possible embodiments thereof, it is to be understood that modifications or improvements may be made thereto without departing from the scope or spirit of the invention.

It should be noted that various changes and modifications to the presently preferred embodiments described herein will be apparent to those skilled in the art. Such changes and modifications may be made without departing from the spirit and scope of the present invention and without diminishing its attendant advantages. It is, therefore, intended that such changes and modifications be included within the present invention.

1. A sampling means for obtaining an organic sample from an animal, the sampling means including separate punch means and housing means, the punch means being adapted to be moved completely through a part of an animal to obtain a sample therefrom and the punch means and the housing means being engageable with each other to contain the sample in the housing means, wherein the sampling means is not associated with an animal tag and wherein the punch means is adapted to be mounted on a first part of an applicator and the housing means is adapted to be mounted on a second part of an applicator, and operation of the applicator causes the punch means to be moved through the part of the animal and to engage with the housing means.

2. A sampling means for obtaining an organic sample from an animal and including a punch means adapted to be securable in use with a first part of an applicator, a housing means adapted in use to be accommodated, or provided, by a second part of the applicator, operation of said applicator in use being adapted to move said punch means through a required part of an animal to extract a sample therefrom, and be receivable within said housing means to enable said housing means, with said punch means and said sample, to be removable from said second part of said applicator, and wherein the sampling means is not associated with an animal tag.

3. A sampling means according to claim 1 or claim 2, further including a removable holding means which is located in said second part of the applicator and the housing means is located in the removable holding means.

4. A sampling means according to claim 1, wherein the punch means includes a tip, and the tip cuts through the part of the animal to extract the sample.

5. A sampling means according to claim 4, wherein the tip engages internally with the housing means.

6. A sampling means according to claim 5, wherein the tip is an interference fit in the housing means.

7. A sampling means according to claim 6, wherein the width of the tip is greater than the internal width of the housing means and the tip and/or the housing means are deformable.

8. A sampling means according to claim 7, wherein the tip and/or the housing are at least partly elastically deformable.

9. A sampling means according to claim 4, wherein the tip has a generally frusto-conical shape.

10. A sampling means according to claim 4, wherein the punch means includes a sample chamber.

11. A sampling means according to claim 10, wherein the sample chamber is in the form of a recess in the punch means.

12. A sampling means according to claim 4, wherein the recess is in an end of the tip.

13. A sampling means according to claim 10, wherein the punch means includes more than one sample chamber.

14. A sampling means according to claim 13, wherein one of the housing means and the punch means has a closure that is openable to permit access to within the housing means.

15. A sampling means according to claim 14, wherein the closure includes a deformable member.

16. A sampling means according to claim 15, wherein the deformable member is elastically deformable so that the closure is self-closing.

17. A sampling means according to claim 15, wherein the closure includes two deformable members.

18. A sampling means according to claim 17, wherein the two deformable members engage each other when the closure is closed and are at least partially separated when the closure is open.

19. A sampling means according to claim 15, wherein the deformable member is inclined towards the inside of the housing.

20. A sampling means according to claim 19, wherein the deformable member is inclined towards the sample chamber.

21. A sampling means according to claim 14, wherein the closure is in the housing means.

22. A sampling means according to claim 21, wherein the closure is at an opposite end of the housing means from where the punch means engages with the housing means.

23. A sampling means according to claim 14, wherein the closure is openable by a laboratory instrument.

24. A sampling means according to claim 23, wherein the laboratory instrument is a pipette.

25. A sampling means according to claim 14, wherein the other of the housing means and the punch means includes a portion adapted to engage with a sample recess in a laboratory tray.

26. A sampling means according to claim 4, wherein the punch means includes an elongate member.

27. A sampling means according to claim 26, wherein the tip is at one end of the elongate member.

28. A sampling means according to claim 27, wherein the elongate member has a mounting formation adapted to enable the punch means to be mounted on an applicator.

29. A sampling means according to claim 28, wherein the mounting formation comprises a recess that is adapted to receive a pin of an applicator.

30. A sampling means according to claim 28, wherein the tip is at the opposite end of the elongate member from the mounting formation.

31. A sampling means according to claim 27, wherein one of the punch means and the housing means includes alignment means to assist in relative alignment of the punch means and the housing means when the punch means engages with the housing means.

32. A sampling means according to claim 31, wherein the alignment means aligns an axis of the punch means with an axis of the housing means.

33. A sampling means according to claim 32, wherein the alignment means includes an alignment formation on the punch means which cooperates with the housing means to assist in the relative alignment.

34. A sampling means according to claim 33, wherein the alignment formation cooperates with an internal side wall of the housing means.

35. A sampling means according to claim 34, wherein the alignment formation is located externally on the elongate member.

36. A sampling means according to claim 35, wherein the alignment formation is on a different section of the elongate member from the tip.

37. A sampling means according to claim 33, wherein a number of alignment formations are provided.

38. A sampling means according to claim 37, wherein there are three alignment formations.

39. A sampling means according to claim 37, wherein the alignment formations are spaced equidistantly around the punch means.

40. A sampling means according to claim 33, wherein the alignment formation extends in a direction along the axis of the punch means that corresponds to the direction of engagement of the punch means with the housing means.

41. A sampling means according to claim 40, wherein the alignment formation extends along the longitudinal axis of the punch means.

42. A method of sampling to provide a sample of organic material from an animal includes:
(i) providing a punch means adapted to be moveable by a first part of an applicator through part of an animal to obtain a sample;
(ii) providing a housing means adapted to be accommodated by a second part of an applicator;
(iii) said punch means and said housing means being engageable together so that the punch means with said sample may be located within said housing means and wherein said housing means and said punch means are able to be removable from said second part of said applicator.

43. A method of sampling for obtaining a sample of organic material from an animal, the method including
(i) mounting a punch means on a first part of an applicator;
(ii) mounting a housing means on a second part of an applicator;
(iii) inserting a part of an animal between the first and second parts of the applicator;
(iv) activating the applicator to cause the punch means to penetrate and pass through the part of the animal to take a sample from the part of the animal, and to engage the housing means so that the sample is located in the housing means;
(v) removing the engaged punch means and housing means from the applicator; and
wherein the sampling method does not occur simultaneously with the application of a tag to the part of the animal.

44. A method according to claim 42, further including mounting a holding means on the second part of the applicator and the housing means is located in or on the holding means.

45. A method according to claim 44, further including removing the holding means from the second part of the applicator either with the engaged punch means and housing means or after removal of the engaged punch means and housing means.

46. A method according to claim 42, further including providing a closure that is openable on one of the punch means and the housing means, and inserting a laboratory instrument through the openable closure to extract at least a portion of the sample for analysis.

47. A method according to claim 46, further including removing the laboratory instrument from the openable closure, whereby the closure is self-closing after removal of the laboratory instrument.

48. A method according to claim 46, wherein the closure is opened by engaging the laboratory instrument with the closure.

49. A method according to claim 46, further including providing the other of the punch means and the housing means with a tray engagement portion and locating the tray engagement portion in a testing tray prior to inserting the laboratory instrument through closure.

50. A method according to claim 46, wherein the closure is provided in the housing means.

51. A method according to claim 46, wherein the laboratory instrument is a pipette.

52. A method according to claim 46, wherein the sample is prepared for analysis in the housing means prior to the portion of the sample being extracted.

53. A method according to claim 52, wherein the punch means covers at least a portion of the first part of the applicator to prevent contact between the first part of the applicator and the part of the animal being sampled.

54. A method according to claim 53, wherein the punch means prevents contact between the first part of the applicator and any animal body fluids exposed as a result of the sampling method.

55. (canceled)
